(19) United States
(12) Patent Application Publication
(10) Pub. No.: US 2024/0329042 A1
(43) Pub. Date: Oct. 3, 2024

(54) CAPTURE REAGENT FOR LATERAL FLOW IMMUNOASSAY

(71) Applicant: IDEXX LABORATORIES, INC., Westbrook, ME (US)

(72) Inventor: Ganesh Raj Panta, Westbrook, ME (US)

(21) Appl. No.: 18/618,910

(22) Filed: Mar. 27, 2024

Related U.S. Application Data
(60) Provisional application No. 63/456,305, filed on Mar. 31, 2023.

(51) Int. Cl. G01N 33/569; G01N 33/543

(52) U.S. Cl. CPC G01N 33/56905; G01N 33/54388; G01N 2469/10

(57) ABSTRACT

Lateral flow immunoassay devices and associated reagents for determining the presence of one or more analytes in liquid samples. A detection zone on a membrane that supports lateral flow includes an immobilized and dried capture reagent including a first antibody conjugated with a first binding partner bound to a second binding partner that is attached to particle.

### FIELD

The present disclosure relates generally to the lateral flow immunoassay devices and associated reagents for determining the presence of one or more analytes in liquid samples.

### BACKGROUND

Lateral flow devices have been used for the detection of analytes in biological, industrial, and environmental samples. Often, however, immunoassays conducted on such devices suffer from a lack of sensitivity desired for many analytes and applications. Lateral flow immunoassay devices that provide increased assay sensitivity while maintaining user convenience for which the devices are disclosed herein.

### SUMMARY

In one aspect, the disclosure is directed to an immunoassay device for determining an antigen in a sample. The device includes (a) a membrane that supports lateral flow of a liquid and comprising a detection zone; and (b) a dried capture reagent in the detection zone comprising a first antibody that specifically binds to the antigen, wherein the first antibody is conjugated with a first binding partner bound to a second binding partner that is attached to particle immobilized in the detection zone. In one embodiment of the disclosure, the first binding partner is biotin and the second binding partner is streptavidin. In addition, the device may include a labeled second antibody that specifically binds to the antigen, wherein the second antibody is diffusively bound in a reagent zone on the immunoassay device. In various embodiments, the label may be an enzyme.

In an example embodiment of the disclosure the immunoassay device may include a housing that supports the membrane. The housing may include a container for a liquid wash reagent and may also include a container comprising a liquid substrate reagent for a substrate for the label.

In another aspect, the disclosure is directed to a kit that includes the immunoassay device of the disclosure and a reagent including a labeled second antibody that specifically binds to the antigen. The label may be an enzyme. The kit may include a housing that supports the membrane and the housing may include a container for a liquid wash reagent and a container for a liquid substrate reagent that includes a substrate for the enzyme.

In a further aspect the disclosure is directed to a method of determining the presence or amount of an antigen in a liquid sample. The method includes adding the sample to a sample application zone of the immunoassay device as described herein and allowing the sample to migrate by lateral flow to the detection zone. A presence or amount of the label in the detection zone may be measured, wherein the presence or amount of the label in the detection provides a signal associated with the presence or amount of the antigen bound in the detection zone.

Still further, in another aspect the disclosure is directed to a method of determining the presence or amount of an antigen in a liquid sample. The method includes forming a mixture by contacting the sample with a reagent comprising a labeled antibody that specifically binds to the antigen, adding the mixture to a sample application zone of the immunoassay device as described herein, allowing the mixture to migrate by lateral flow to the detection zone, and detecting the presence or amount of the label in the detection zone, wherein the presence or amount of the label in the detection provides a signal associated with the presence or amount of the antigen bound in the detection zone.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure. No attempt is made to show structural details of the disclosure in more detail than may be necessary for a fundamental understanding of the disclosure and various ways in which it may be practiced.

FIG. 1, Panels A and B, show optional formats for a lateral flow test strip of the disclosure.

FIG. 2 shows gray scale images of detection zones from lateral flow membranes according to the disclosure for a series of clinical samples known positive for Whipworm antigen that may also contain Hookworm antigen and Roundworm antigen. Signal minus background (S-B) was measured by densitometry and shown for each detection zone (some positive sample spots may not be visible in the Figure as a result of the conversion of the images to grayscale). A legend of the location of capture antibodies immobilized in the detection zone is also shown. Concentrations of the antigens in the clinical samples were determined by ELISA and the optical density for each ELISA sample (ELISA OD) is shown.

FIG. 3 shows gray scale images of detection zones from lateral flow membranes according to the disclosure that were used in the analysis of a series of known concentrations of recombinant Hookworm, Roundworm and Whipworm antigens. Signal minus background (S-B) was measured by densitometry and shown for each detection zone. Arrows point to sample spots that may not be visible in the Figure as a result of the conversion of the images to grayscale (in addition, other faint sample spots, while present, may not be clearly visible in the Figure as the result of reproduction). A legend of the location of capture antibodies immobilized in the detection zone is also shown.

FIG. 4 shows the summary results of FIG. 3 showing immunoassays on lateral flow membranes according to the disclosure at known concentrations for recombinant Hookworm antigen, Roundworm antigen and Whipworm antigen.

FIG. 5 shows gray scale images of detection zones from lateral flow membranes according to the disclosure that were used in the analysis of a samples known to be negative for Roundworm, Hookworm and Whipworm antigens. No positive signals were detected.

### DESCRIPTION

The disclosure is directed to an immunoassay device including reagents for use in the determination of analytes in liquid samples. The disclosure provides a lateral flow membrane with a non-diffusively bound capture reagent that includes an antibody that specifically binds to an antigen in a sample, wherein the antibody (or other binding partner for an analyte) is biotinylated and attached to a streptavidin-coated particle that is immobilized on the membrane.

Before describing the present invention in detail, a number of terms will be defined. As used herein, the singular forms “a,” “an”, and “the” include plural referents unless the context clearly dictates otherwise.

The term “analyte,” as used herein, generally refers to the substance, or set of substances, in a sample that are detected and/or measured. In some embodiments, the analyte is an antigen.

The term “antibody,” as used herein, generally refers to a glycoprotein produced by B lymphocyte cells in response to exposure to an antigen and binds specifically to that antigen. The term “antibody” is used in its broadest sense and specifically covers monoclonal antibodies (including full length monoclonal antibodies), polyclonal antibodies, multispecific antibodies (e.g., bispecific antibodies), and antibody fragments so long as they exhibit the desired biological activity. Antibodies may be naturally produced or engineered by methods well known to those of skill in the art.

The term “antibody fragment,” as used herein, refers to a portion of a full length antibody, generally the antigen binding or variable domain thereof. For example, antibody fragments may include Fab, Fab′, F(ab′)2, and Fv fragments; diabodies; linear antibodies; single-chain antibody molecules; and multi-specific antibodies from antibody fragments.

The term “antigen,” as used herein, generally refers to a substance that is capable, under appropriate conditions, of reacting with an antibody specific for the antigen.

The term “sample,” as used herein, generally refers to a sample of tissue, excreted product, or fluid from a human or animal including, but not limited to whole blood, plasma, serum, spinal fluid, lymph fluid, abdominal fluid (ascites), the external sections of skin, respiratory, intestinal and genitourinary tracts, tears, saliva, urine, blood cells, tumors, organs, tissue, feces and in vitro cell culture constituents. Samples may also include industrial or environmental samples that require analysis. Samples may require mechanical or chemical processing prior to analysis (e.g, separation, filtering, centrifugation, chemical modification of sample constituents). As used herein, samples include both raw samples and/or processed samples.

The term “immunoassay,” as used herein, generally refers to a test that employs antibody and antigen complexes to generate a measurable response. An “antibody: antigen complex” may be used interchangeably with the term “immuno-complex.” Immunoassays, in general, include noncompetitive immunoassays, competitive immunoassays, homogeneous immunoassays, and heterogeneous immunoassays. In “competitive immunoassays,” unlabeled analyte (or antigen) in the test sample is measured by its ability to compete with labeled antigen in the immunoassay. The unlabeled antigen blocks the ability of the labeled antigen to bind because the binding site on the antibody is already occupied. In “competitive immunoassays,” the amount of antigen present in the test sample is inversely related to the amount of signal generated from the label. Conversely, in “noncompetitive immunoassays,” also known as “sandwich” immunoassays, the analyte is bound between two highly specific antibodies to form a complex and the amount of antigen is directly proportional to the amount of signal associated with the complex. Immunoassays that require separation of bound antibody: antigen complexes are generally referred to as “heterogeneous immunoassays,” and immunoassays that do not require separation of antibody: antigen complexes are generally referred to as “homogeneous immunoassays.” One of skill in the art would readily understand the various immunoassay formats.

The term “label,” as used herein, refers to a detectable compound, which can be conjugated directly or indirectly (e.g., via covalent or non-covalent means, alone or encapsulated) to an antibody or analogs of the disclosure. The label may be detectable by itself (e.g., chemiluminescent dye, fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current disclosure could be, but is not limited to: alkaline phosphatase; glucose-6-phosphate dehydrogenase (“G6PDH”); horse radish peroxidase (HRP); chemiluminescers such as isoluminol, fluorescers such as fluorescein and rhodamine compounds; ribozymes; and dyes. The label produces a signal that may be detected by means such as detection of electromagnetic radiation or direct visualization, and that can optionally be measured.

The terms “membrane” or “matrix” as used herein, refer to a non-aqueous matrix to which the components of an immunoassay, including the reagents of the disclosure to be diffusively or non-diffusively bound. Examples of membranes and matrices include supports formed partially or entirely of glass (e.g., controlled pore glass), synthetic and natural polymers, polysaccharides (e.g., agarose), polyacrylamides, polystyrene, polyvinyl alcohols and silicones that are formed in to chromatographic that will allow reagents that become bound to be washed or separated from unbound materials. In some embodiments, the membranes or matrices can be porous.

The term “particle” or “particles” in connection with the disclosure include, for example, particles of latex, polystyrene, or of other support materials such as silica, agarose, ceramics, glass, polyacrylamides, polymethyl methacrylates, carboxylate modified latex, melamine, and Sepharose. The particles will vary in size from about 0.1 microns to about 100 microns, for example about 0.1, 0.5, 1.0, 5, 10, 20, 30, 40 50, 60, 70, 80 90 or 100 microns. In particular, useful commercially available materials include carboxylate modified latex, cyanogen bromide activated Sepharose beads, fused silica particles, isothiocyanate glass, polystyrene, and carboxylate monodisperse microspheres.

Particles suitable for use in the present disclosure are capable of attachment to other substances such as derivatives, linker molecules, or proteins. The capability of the particles to be attached to other substances can result from the particle material as well as from any surface modifications or functional groups added to the particle. The particles can be functionalized or be capable of becoming functionalized in order to covalently or non-covalently attach proteins, linker molecules or derivatives as described herein. Suitable functional groups include, for example, amine, biotin, streptavidin, avidin, protein A, sulfhydryl, hydroxyl and carboxyl.

The term “multiplex” refers to an immunoassay can detect more than one antigen in a single sample of a single experiment.

The term “capture reagent” refers to complex that comprises a binding partner for an analyte (e.g., an antibody that binds antigen), which is immobilized to a particle through the interaction of separate set of binding partners (e.g., biotin and streptavidin). For example, an antibody conjugated to biotin may be bound to particle that is functionalized with streptavidin.

All patent references identified herein are incorporated by reference in their entirety.

Turning now to the various aspects of the disclosure, an immunoassay device for determining one or more analytes in a sample is described herein. As an example, the device includes a membrane that supports lateral flow of a liquid and includes one or more immobilized capture reagents that bind to an analyte in the sample.

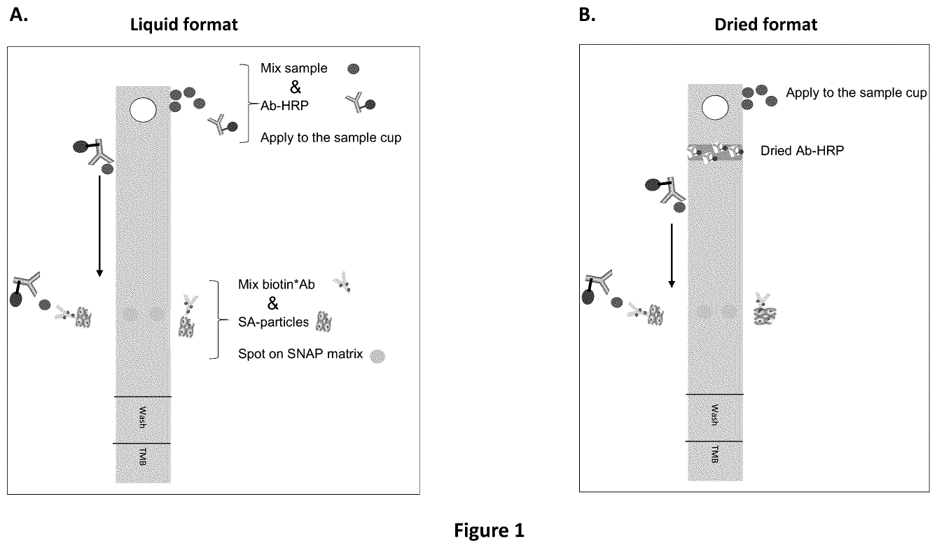

In an example embodiment of the disclosure, the membrane supports lateral flow of the sample and, optionally, other liquid reagents that are applied to the membrane. In some embodiments, the membrane includes a sample application zone and a detection zone. Sample applied to the membrane in the sample application zone migrates through the membrane towards the detection zone where it encounters an immobilized capture reagent as described herein. In one embodiment that is shown in FIG. 1, Panel A, the sample is mixed, before the sample is applied to the membrane, with a detection reagent that includes a labeled binding partner for the analyte that binds to analyte in the sample. In another embodiment that is shown in FIG. 1, panel B, the detection reagent is dried in a reagent zone on the membrane such that the sample solution solubilizes the detection reagent and transports it along with the analyte in the sample, if any, towards the detection zone.

The membrane of the disclosure may or may not be contiguous as long as there is fluid communication between a portion of the membrane having a sample application zone and a portion of the membrane having a detection zone as described herein. In an alternative embodiment, the reagent zone may itself be present on a separate membrane that the liquid sample encounters before the sample contacts a portion of the membrane having the detection zone.

In various embodiments of the disclosure, the capture reagent includes a binding partner for the analyte (e.g., an anti-antigen antibody) that binds the analyte (e.g., an antigen) in the sample, wherein the binding partner for the analyte is bound to a particle that is immobilized in the detection zone. In one aspect, the antibody is bound to the particle through the interaction of a separate pair of binding partners, one attached to the binding partner for the analyte and the other attached to the particle. Examples of sets of separate binding partners include (a) biotin and avidin (streptavidin), (b) digoxigenin and anti-digoxigenin, (c) nucleic acids that hybridize, and (d) an anti-species antibody (for example, anti-mouse) and a species antibody (IgG) as the first antibody. One of binding partners of the separate set of binding partners can be coupled to the binding partner for analyte and the other may be attached to the particle by techniques known to those of skill in the art. In one example embodiment, the binding partner for the analyte on the capture reagent is a biotinylated anti-analyte antibody that is attached to a streptavidin-coated particle through the biotin-streptavidin interaction. The capture reagent may be deposited in a liquid to the detection zone and allowed to dry, which immobilizes the capture reagent to the membrane in the detection zone.

The detection reagent includes a labeled binding partner for the analyte. In various embodiments when the analyte binding partner of the detection reagent and the analyte binding partner of the capture reagent are antibodies, the detection antibody and the capture antibody may be the same or different depending on the available epitopes on the antigen of interest in the sample. The label present in the detection zone can be detected. This can be done, for example, visually, or by spectrophotometry or densitometry. For example, the label may be an enzyme that provides a visual signal upon contact with a substrate. An example enzyme is horseradish peroxidase (HRP) for which substrates are known. In this embodiment, the detection reagent of the disclosure may include, for example, the anti-antigen antibody bound to HRP. The reagent may be added to the sample or may be dried in a reagent zone of the membrane as described herein.

Binding partners other than antibodies specific for antigens are known. Examples include nucleic acids (DNA/DNA, DNA/RNA etc.), proteins and other peptides that are known to specifically bind to various molecules in biological, industrial and environmental samples.

The membrane used in a lateral flow device is made of materials that support lateral flow of liquids. The suitable materials include fibrous mats composed of synthetic or natural fibers (e.g., glass or cellulose-based materials or thermoplastic polymers, such as, polyethylene, polypropylene, or polyester); sintered structures composed of particulate materials (e.g., glass or various thermoplastic polymers); or cast membrane films composed of nitrocellulose, nylon, polysulfone or the like (generally synthetic in nature). The matrix may also be composed of sintered, fine particles of polyethylene, commonly known as porous polyethylene, such as sintered polyethylene beads. Such material may have (a) a density of between about 0.30 to about 0.60 grams per cubic centimeter, for example, 0.35 and 0.55 grams per cubic centimeter, (b) a pore size of between about 1 and 50 microns, or between about 5 and 40 microns, and (c) a void volume of between about 25 and 75 percent or about 40 and 60 percent. For example, material density may be 0.30, 0.35, 0.40, 0.45, 0.50, 0.55 or 0.60 grams per cubic centimeter. Pore size may be 1, 2, 3, 4, 5, 10, 15, 20, 25, 30, 35, 40, 45 or 50 microns. Void volume may be about 25, 30, 35, 40, 45, 50, 55, 60, 65 or 70 percent. Particulate polyethylene composed of cross-linked or ultra-high molecular weight polyethylene may also be used. Example matrices include FUSION 5™ matrix available from Whatman, Inc., and various sintered plastic materials from Porex Corporation. The matrix may be selected in view of the samples to be applied to the matrix and other parameters that are known to those of skill in the art.

In aspects of the disclosure, the lateral flow membrane of the device is supported in a housing. In addition to supporting the membrane, the housing may include a container holding a liquid wash reagent. In addition, the housing may include a container holding a liquid containing the substrate for the label when it is an enzyme (a “substrate reagent”). Embodiments of a housing including onboard wash and substrate reagents are shown, for example, in U.S. Pat. No. 5,627,010 and are sold under the SNAP® brand by IDEXX Laboratories, Inc. Briefly, upon addition of the sample to such a device, the sample migrates to the detection zone wherein the antigen, if present, binds the capture reagent. The sample liquid carries the labeled second antibody that was added to the sample or present in a reagent zone. When the antigen is present in the sample, the label of the detection reagent becomes bound in the detection zone through the interaction of the capture reagent, sample antigen and second antibody (i.e., immunoassay sandwich format). An operator can activate the device to release the wash reagent and the substrate reagent onto lateral flow membrane such that migration of the wash reagent removes unbound reactants from the detection zone and the substrate produces a signal by reacting with the enzyme label attached to the detection reagent. The signal can be read by the operator for example by visual or spectrophotometric means. In some embodiments, the wash and substrate reagents are the same reagent. If the label does not require a substrate for detection (e.g., a fluorescent label), the device would not require the substrate reagent.

In a related aspect, the device may be used in a competitive assay format. Accordingly, a capture reagent is bound in the detection zone and an analyte-label conjugate may be mixed with the sample or present in the reagent zone on the device as described herein. Once the sample added to the device, the labeled conjugate will compete with analyte in the sample for binding to the capture reagent in the detection zone. The absence of signal from the label in the detection zone indicates the presence of analyte in the sample.

Another aspect of the disclosure is directed to a multiplex device that has the ability to determine more than one analyte in the sample. In this aspect, one or more detection reagents can bind to one or more respective analytes. The label on one or more detection reagents binding to one or more respective analytes can be the same or different as long as the labels may be differentiated from each other. For instance, when the capture reagents for different analytes in the sample are located in distinct areas of the detection zone, the same label on each detection reagent may be used and associated with the corrected antigen due to the location of the label in the detection zone. If all of the capture reagents are located in an area of the detection zone where the locations of the capture reagents cannot be distinguished from one another, the labels may be differentiated by their emitted wavelengths. For example, when the labels are fluorescent or chemiluminescent, each label may emit light at a different wavelength, which typically is detected with a spectrophotometer.

In yet another aspect, the disclosure is directed to a method for determining the presence or amount of one or more antigen in a sample. The method includes adding a sample suspected of containing the analyte(s) to the sample application zone of a device according to the disclosure and allowing the sample to migrate by lateral flow to the detection zone where it encounters immobilized capture reagent(s). Detection reagent(s) may be added to the sample or may be dried in a reagent zone on the membrane as described herein. An operator can detect the presence or amount of the label in the detection zone, wherein the presence or amount of the label provides a signal associated with the presence or amount of the analyte in the sample. As noted above, the device may include a housing supporting the membrane and may include a container(s) for a wash reagent and a substrate reagent such that, upon activation of the device, the wash reagent washes the detection zone to remove unbound reactants. The substrate for the label can provide a detectable signal if the sample contains an antigen.

Some embodiments of the disclosure are directed to using one or more reagents of the disclosure for detecting fecal antigens including Hookworm, Roundworm and Whipworm in a stool sample that has been processed for application to the device in liquid form. In example embodiments, the detection reagent is a detection antibody for one of the fecal antigens (described in Examples below) conjugated to enzyme such as HRP. One or more capture reagents including an anti-Hookworm antigen antibody, and anti-Roundworm antigen antibody and/or an anti-Whipworm antigen antibody is immobilized in the detection zone of the device as described herein.

### EXAMPLES

### Description of Assays

As a model system, a sintered matrix immunoassay device format (SNAP® device, IDEXX Laboratories, Inc.) that utilizes a reversible flow on the matrix (see, e.g., U.S. Pat. No. 5,726,010A) was used to determine the sensitivity and specificity of an immunoassay for fecal antigens using a biotinylated antibody immobilized to a streptavidin-coated particle as a capture reagent. The matrix used was a porous polyethylene pad made by sintering polyethylene particles (Porex Corporation). Results show that the assay sensitivity is as high as the standard ELISA assay with an absence of non-specific binding. Results also indicate that the assay can be multiplexed for the simultaneous detection of multiple antigens.

### Materials and Methods

The following describes procedures to prepare a capture reagent (streptavidin coated particle with biotinylated antibody) and sample mixed with an antibody conjugated with HRP in liquid or dry format, all of which are used on a SNAP® reversible flow assay device.

### Streptavidin-Biotinylated Antibody Particle (Capture Reagent) Preparation Protocol

1.5 mL of 1% w/v and 0.4-0.69 μm nominal size Streptavidin coated particle (SVP-05-10 Spherotech) was centrifuged at 17,000 rpm for 10 minutes and resuspended in 750 μL of carbonate buffer (10 mM at pH 10.5). The process was repeated two times and the pellet was finally resuspended in 750 μL of carbonate buffer (10 mM at pH 10.5) to make a 2% w/v particle containing solution. The solution was sonicated until no aggregation was observed under the microscope. Next, anti-antigen antibodies (anti-Whipworm antigen, anti-Roundworm antigen, and anti-Hookworm antigen) (antibodies prepared in-house) were biotinylated by conjugating the antibodies to sulfo-NHS-LC-Biotin. The antibody conjugates were mixed with the streptavidin coated parties (Spherotech, Inc.) and sonicated until no aggregation was observed. The mixture was incubated at room temperature on an end-to-end rotation for 40 minutes, and then centrifuged at 17,000 rpm for 10 minutes. The supernatant was removed and the streptavidin-biotinylated antibody particles were resuspended in appropriate volume of solution to a achieve desired of % w/v concentration. The solid particle percentage concentration was measured by factoring the dilution of the original stock of the streptavidin particles.

The particle solution was spotted/deposited directly onto a SNAP® sintered matrix in a “detection zone” at a desired % concentration in a spotting buffer (10 mM carb-bicarb buffer, pH 10, 2.5% Sucrose, 0.05% Tween-20). The volume of the spot was 1 μL. The spotted strip was allowed to dry for 10 minutes at 30° C. and stored with a desiccant in a foil bag. The detection zone can be prepared with capture reagents for one or more antigens.

### Sample and Secondary Antibody Conjugated HRP (Detection Reagent) Preparation

Liquid format: 350 μL of sample was prepared by mixing extraction buffer (pH 10.5) with fecal mass followed by centrifugation. The secondary antibodies (detection antibodies) binding to a different epitopes on the antigens were conjugated to horseradish peroxidase (HRP) by utilizing SMCC chemistry. To conjugate the antibody with HRP, a mixture of conjugate buffer (10 mM PBS Buffer/2 mM EDTA), 0.5M EDTA, and antibody at a concentration of 5 mg/mL was prepared in a tube. DTT at a concentration of 5 mM was added to the mixture to reduce the antibody, which was then incubated at 18-27° C. for 30 minutes and desalted using a G-25 column. The reduced antibody was then combined with HRP-SMCC in a 1:1 to 1:4 molar ratio and incubated for 2 hours at 18-27° C. Any unreacted sulfhydryl groups were capped using 0.5 mM L-Cys. Finally, the conjugated antibody-HRP was characterized through SEC prior to use in the SNAP.

The antibody-HRP conjugates were added directly to and mixed with the sample (see FIG. 1, panel A). The sample mixture (350 μL) was then loaded onto a sample cup of a SNAP® device having the matrix as described above.

Dry format: in another embodiment, a dry format in which the secondary antibody conjugated to HRP (can be dried onto a strip (see FIG. 1, panel B) by first mixing the conjugate with a solution containing 20-25% sucrose/trehalose and striping the mixture onto the matrix. The matrix is allowed to dry at 37° C. The dried matrix may be stored with a desiccant in a foil bag.

The sample can be prepared as above, except without mixing with the antibody-HRP conjugate, and loaded directly onto the sample cup. As the sample passes the dried conjugate on the strip, the sample will contact and solubilize the conjugate.

### Sample Analysis on SNAP®

The spotted strip prepared as above may be installed into the SNAP® device containing an absorbent block and wash and substrate reservoirs. The appropriate sample (depending on liquid/dry format) may be loaded onto the sample cup. Once the sample flow reaches the activation circle (allowing sufficient time for antigen binding to antibody at the spot), the device was activated to discharge the wash and substrate solution (e.g., tetramethylbenzidine (TMB) sequentially to reverse the direction of the liquid flow on the strip. After about 5 minutes, a blue color will be visible in the detection zone (as a result of oxidation of the TMB substrate). While not required, the color development may be discontinued with the addition of sodium azide, but this step is not required. Color development indicates a sample positive for antigen.

### Assay Spot Density Measurement

For quantitative measurements to determine assay sensitivity, a densitometer was used to measure the optical density of the assay spot as well as the background density of the membrane. The background density was subtracted from the assay spot density to calculate a sample-background (S-B).

### Example 1: Detection of Fecal Antigen

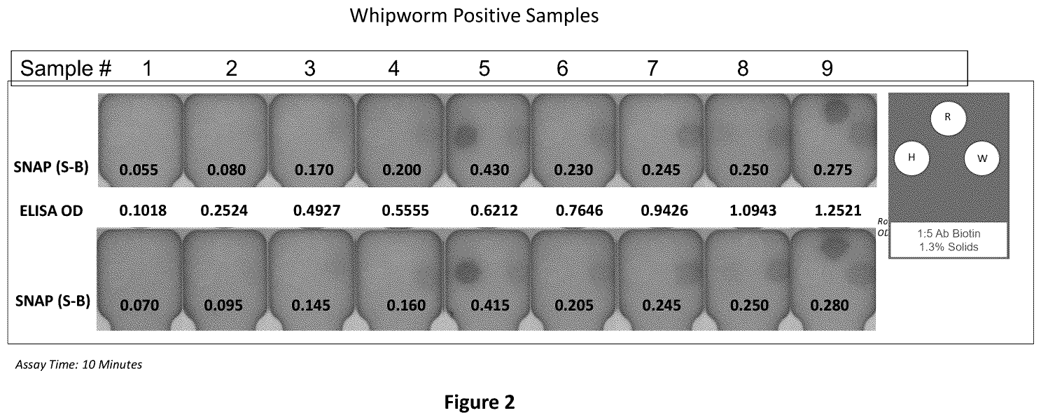

As shown in FIG. 2, an assay on a SNAP® device with spotted with capture reagent (1 μL of 1.3% w/v streptavidin particles) described above (particle-streptavidin-biotin-antibody) was able to detect Whipworm antigen in a series of clinical fecal samples that were identified as positive with a standard ELISA assay at OD>0.1. The detection zones shown in FIG. 2 also contained capture reagent for Roundworm and Hookworm antigens. Secondary antibody-HRP conjugate (Anti-hookworm antibody=2 ug/mL, anti-roundworm Ab=4.5 ug/mL, anti-whipworm Ab=3.0 ug/mL) was added to the sample prior to the addition of the sample to the device as described above. Sample solution was added to the device without incubation.

The assay was sensitive to Whipworm antigen positive clinical samples (that may have also contained Hookworm and/or Roundworm antigen) at concentrations as low as 0.5 ng/ml which was comparable to ELISA's sensitivity at 0.1-0.8 ng/mL. Furthermore, as shown in FIG. 2, Whipworm antigen was detected without non-specific binding or cross reactivity with samples that were also positive for Hookworm and Roundworm antigen. The absence of cross reactivity becomes more evident when the sample is positive only for whipworm, as no indications of roundworm or hookworm were detected.

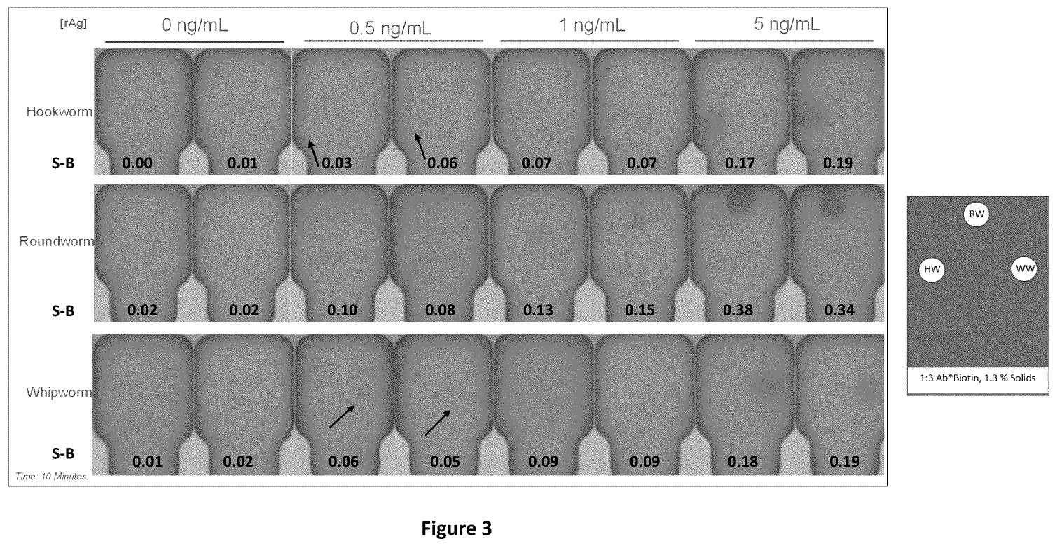

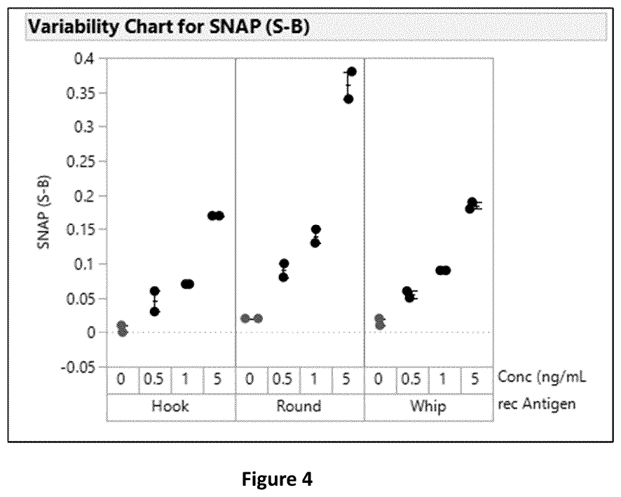

FIG. 3 similar results for detection zones with known concentrations for sample antigen (0 ng/mL, 0.5 ng/mL, 1 ng/mL and 5 ng/mL). The results are summarized in FIG. 4.

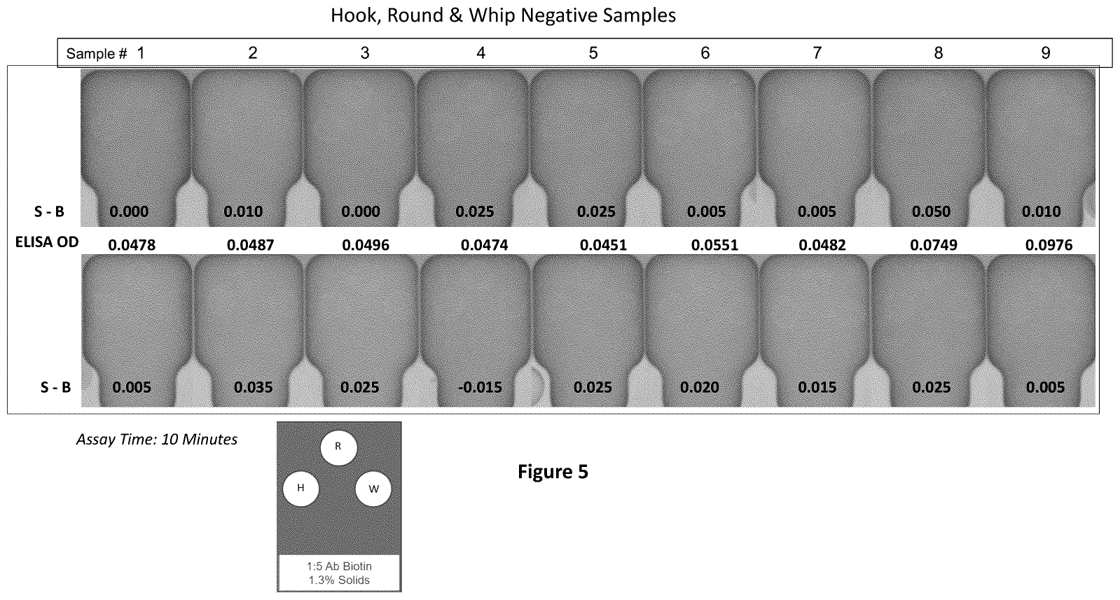

FIG. 5 shows detection zones from samples negative for the three antigens (consistent with ELISA OD<0.1).

### Example 2: Variation on Assay Parameters

In order to assess reaction conditions, two fecal masses at 0.1 and 0.3 g/mL were tested with different incubation conditions for sample and Ab-HRP (N=9-14 for positive samples and 26-30 for negative samples). As shown in Table 2, low fecal mass (0.1 g/mL) and incubation of fecal sample with Ab-HRP produced more consistent results with ELISA/less false results than no incubation. At high fecal mass 0.3 g/mL, room temperature (RT) incubation was more accurate than 50° C. incubation.

TABLE 2

| | 0.1 g fecal mass/mL extraction buffer | | | | 0.3 g fecal mass/mL extraction buffer | | | |
|---|---|---|---|---|---|---|---|---|
| | Incubation | | | | | | | |
| | No incubation | | 3 min at 50° C. | | No incubation | | 3 min at 50° C. | |
| | Antigens | | | | | | | |
| | Negative | Positive | Negative | Positive | Negative | Positive | Negative | Positive |
| Hookworm | 90% | 100% | 89.7% | 100% | 93.1% | 100% | 89.7% | 90.9% |
| Roundworm | 100% | 91.7% | 100% | 100% | 100% | 100% | 96.2% | 92.9% |
| Whipworm | 100% | 100% | 100% | 100% | 100% | 100% | 100% | 90.9% |

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

What is claimed is:

1. An immunoassay device for determining an antigen in a sample, comprising
a membrane that supports lateral flow of a liquid and comprising a detection zone; and
a dried capture reagent in the detection zone comprising a first antibody that specifically binds to the antigen, wherein the first antibody is conjugated with a first binding partner bound to a second binding partner that is attached to particle immobilized in the detection zone.

2. The device of claim 1, wherein the first binding partner is biotin and the second binding partner is streptavidin.

3. The immunoassay device of any of claim 1, further comprising a labeled second antibody that specifically binds to the antigen, wherein the second antibody is diffusively bound in a reagent zone on the immunoassay device.

4. The immunoassay device of claim 3, wherein the label is an enzyme.

5. The immunoassay device of claim 1, further comprising a housing that supports the membrane.

6. The immunoassay device of claim 5, wherein the housing comprises a container comprising a liquid wash reagent.

7. The immunoassay device of claim 6, wherein the housing further comprises a container comprising a liquid substrate reagent comprising a substrate for the label.

8. A kit comprising the immunoassay device of claim 1 and a reagent comprising a labeled second antibody that specifically binds to the antigen.

9. The kit of claim 8, wherein the immunoassay device further comprising a housing that supports the membrane.

10. The kit of claim 9, the housing comprises a container comprising a liquid wash reagent.

11. The kit of claim 9, wherein the label is an enzyme.

12. The kit of claim 11, wherein the housing further comprises a container comprising a liquid substrate reagent comprising a substrate for the enzyme.

13. A method of determining the presence or amount of an antigen in a liquid sample, the method comprising,
adding the sample to a sample application zone of the immunoassay device of claim 3,
allowing the sample to migrate lateral flow to the detection zone; and
detecting the presence or amount of the label in the detection zone, wherein the presence or amount of the label in the detection provides a signal associated with the presence or amount of the antigen bound in the detection zone.

14. The method of claim 13, wherein the immunoassay device further comprises a housing supporting the membrane and comprising a container comprising a wash reagent, wherein the method comprising washing the detection zone with the wash reagent.

15. The method of claim 14, wherein the immunoassay device further comprises a housing supporting the membrane and comprising a container comprising a wash reagent and a container comprising a substrate of the label, wherein the method comprises (i) washing the reactive zone by adding the wash reagent to the membrane, and (2) adding the substrate to the reactive zone.

16. A method of determining the presence or amount of an antigen in a liquid sample, the method comprising,
forming a mixture by contacting the sample with a reagent comprising a labeled antibody that specifically binds to the antigen;
adding the mixture to a sample application zone of the immunoassay device of claim 1,
allowing the mixture to migrate by lateral flow to the detection zone; and
detecting the presence or amount of the label in the detection zone, wherein the presence or amount of the label in the detection provides a signal associated with the presence or amount of the antigen bound in the detection zone.

17. The method of claim 16, wherein the immunoassay device further comprises a housing supporting the membrane and comprising a container comprising a wash reagent and a container comprising a substrate for the label, wherein the method comprises (i) washing the reactive zone by adding the wash reagent to the membrane, and (2) adding the substrate to the reactive zone.
